(19) United States
(12) Patent Application Publication
(10) Pub. No.: US 2024/0050446 A1
(43) Pub. Date: Feb. 15, 2024

(54) DEXAMETHASONE FOR TREATMENT OF BLEPHARITIS

(71) Applicant: SUN PHARMACEUTICAL INDUSTRIES LIMITED, Mumbai, Maharashtra (IN)

(72) Inventor: Lyle BOWMAN, Pleasanton, CA (US)

(73) Assignee: SUN PHARMACEUTICAL INDUSTRIES LIMITED, Mumbai, Maharashtra (IN)

(21) Appl. No.: 18/278,583

(22) PCT Filed: Feb. 24, 2022
(86) PCT No.: PCT/IB2022/051642

§ 371 Date: Aug. 23, 2023

(30) Foreign Application Priority Data
Feb. 25, 2021 (IN) 202121008105

(51) Int. Cl. A61K 31/573; A61P 27/02; A61F 9/00; A61K 9/00

(52) U.S. Cl. CPC A61K 31/573; A61P 27/02; A61F 9/0008; A61K 9/0048

(57) ABSTRACT

The present invention relates to a method of treating blepharitis comprising administering to the affected eye of a subject an effective amount of an active ingredient in an ophthalmically acceptable vehicle for a sufficient period of time to treat blepharitis. The active ingredient comprises a glucocorticoid in an ophthalmically acceptable vehicle, such as DuraSite® 2. Additionally, the present invention discloses a kit that includes: (a) a composition comprising about 0.1% by weight dexamethasone in an ophthalmically acceptable vehicle and, optionally, (b) instructions for using the composition for the treatment of blepharitis.

### FIELD OF THE INVENTION

The present invention relates to methods for treating ocular inflammatory diseases and, more specifically to methods for treating blepharitis.

### BACKGROUND OF THE INVENTION

This invention relates generally to methods for treating ocular inflammatory diseases and, more specifically to methods for treating blepharitis.

Blepharitis is an ocular disease characterized by inflammation of the eyelid margins. Blepharitis may cause redness of the eyes, itching and irritation of the eyelids in one or both eyes. The pathophysiology of blepharitis is a complex combination of any number of factors, including abnormal lid-margin secretions, bacterial organisms, and abnormalities of the tear film. Other causative agents of blepharitis can be fungal or viral in origin including, for example, herpes simplex and varicella zoster. Blepharitis can appear along with various dermatological conditions including, for example, seborrheic dermatitis, rosacea, and eczema.

Blepharitis occurs in two main forms. The first type, anterior blepharitis, affects the outside front of the eyelid near the eyelashes. The two most common causes of anterior blepharitis are Staphylococcus bacterial infection and seborrheic dermatitis. The second type, posterior blepharitis, affects the inner eyelid and can be caused by problems with the meibomian glands. Two skin disorders that commonly cause this form of blepharitis are acne and rosacea, which leads to red and inflamed skin, and seborrheic dermatitis.

Blepharitis has a strong tendency to recur and if left untreated can lead to conjunctivitis and the eyelids can begin to ulcerate in some circumstances. It is most commonly treated, although not cured, via a thorough hygiene regimen that helps remove crusts and some bacterial organisms. Under duress to prevent or treat ulcerative blepharitis, pharmaceutical interventions have utilized mixtures of anti-inflammatory agents in conjunction with topical or systemic antibacterial agents.

With the ubiquitous usage of antibacterial agents, there is the risk that organisms will develop drug resistance.

Thus, there exists a need for improved treatments for blepharitis. The present invention satisfies this need and provides related advantages as well.

### SUMMARY OF THE INVENTION

In some aspects, embodiments of the present invention relate to a method of treating blepharitis that includes administering to the affected eye of a subject an effective amount of an active ingredient in an ophthalmically acceptable vehicle for a sufficient period of time to treat blepharitis. The active ingredient may comprise, consist, or consist essentially of a glucocorticoid, such as dexamethasone, which is provided in an ophthalmically acceptable vehicle. The vehicle may be DuraSite® 2.

### DETAILED DESCRIPTION OF THE INVENTION

Aspects of the present disclosure relate to compositions comprising dexamethasone in a vehicle, such as DuraSite® 2, methods of using the composition, methods of preparing the composition, and kits or delivery devices containing the composition. The vehicle may be DuraSite® 2. Accordingly, the vehicle may be a composition disclosed in U.S. Pat. No. 8,501,800 to Bowman et al., the entire contents of which are incorporated herein by reference. The vehicle may also be or include a different pharmaceutically acceptable vehicle.

The concentration of dexamethasone in the composition is not particularly limited, but in some embodiments may be 0.1% w/w of dexamethasone in a vehicle. The compositions may be used to treat blepharitis and/or ocular pain. The blepharitis may be active blepharitis and/or symptomatic blepharitis, while ocular pain may be post-operative and/or in a subject undergoing ocular surgery.

In certain embodiments, the compositions do not include an active other than dexamethasone.

In certain embodiments, the compositions do not include an antibiotic.

Certain methods of treating blepharitis are disclosed in U.S. Pat. No. 10,201,548, the entire contents of which are incorporated herein by reference.

As used herein, the term “ocular infective condition” includes those conditions, associated with ocular inflammation, pain or both. The ocular infective condition is selected from blepharitis, post-operative ocular inflammation, post-operative ocular pain, prevention of ocular pain in subjects undergoing ocular surgery and combinations thereof.

As used herein, the term “blepharitis” includes all types of ocular disease characterized by inflammation of the eyelid margins, including the broad categories of anterior blepharitis and posterior blepharitis. The term encompasses blepharitis characterized by its pathophysiological origins, including for example, staphylococcal, seborrheic, mixed staphylococcal and seborrheic, and meibomian gland dysfunction (MGD). Pathophysiological origins for which a glucocorticoid is contraindicated are not encompassed by the term and include the less common viral and fungal forms of blepharitis.

As used herein, the term “post-operative ocular inflammation” or “post-surgery ocular inflammation” includes inflammations resulting from photorefractive surgery, cataract removal surgery, LASIK surgery, intraocular lens implantation or radial keratotomy.

As used herein, the term “ocular pain”, unless otherwise specified, it includes post-operative ocular pain and ocular pain in subject/patient undergoing ocular surgery, which includes ocular pain resulting during or after photorefractive surgery, cataract removal surgery, LASIK surgery, intraocular lens implantation or radial keratotomy.

As used herein, “administering to the eye of a subject” means administering the active ingredient in an ophthalmically acceptable vehicle drop directly to the eye and/or in the eyelid margins, for example, in the form of an eye drop, such administration techniques being familiar to persons skilled in the art.

As used herein, “an effective amount” when used in connection with treating blepharitis and/or ocular pain, is intended to qualify the amount of a glucocorticoid used in the treatment thereof. This amount will achieve the goal of preventing, reducing, or eliminating blepharitis and/or ocular pain. In one embodiment, an effective amount includes from about 0.01 (mg/mL or μg/mL) to 100 per dose in one embodiment. An “effective amount” can include a dose regimen once per day, twice per day, thrice per day, and so on.

As used herein an “ophthalmically acceptable vehicle” is one which allows delivery of an active ingredient to treat blepharitis and/or ocular pain without deleterious effects on the eye. An ophthalmically acceptable vehicle is one that can maintain proper intraocular pressure and provide solutions that are either isotonic, mildly hypotonic, or mildly hypertonic. To maintain such conditions one can include various non-ionic osmolality-adjusting compounds such as polyhydric alcohols, including, for example, glycerol, mannitol, sorbitol, or propylene glycol. Alternatively, osmolality adjusting compounds can include ionic salts such as sodium or potassium chloride.

An ophthalmically acceptable vehicle can also include buffers to adjust to an acceptable pH, which can range from about 3 to 7.4. Such buffer systems include, for example, acetate buffers, citrate buffers, phosphate buffers, borate buffers and mixtures thereof. Specific buffer components useful in the present invention include citric acid/sodium citrate, boric acid, sodium borate, sodium phosphates, including mono, di- and tri-basic phosphates, such as sodium phosphate monobasic monohydrate and sodium phosphate dibasic heptahydrate, and mixtures thereof. It should be noted that any other suitable ophthalmically acceptable buffer components can be employed to maintain the pH of the ophthalmic formulation so that the ophthalmic formulation is provided with an acceptable pH, and the foregoing buffer components are merely exemplary of such buffer components.

An ophthalmically acceptable vehicle includes an aqueous suspension that has a first viscosity and includes (i) about 0.1% to about 6.5% by weight of a carboxyl-containing polymer; (ii) a second polymer that allows the carboxyl-containing polymer to remain suspended, wherein upon contact with tear fluid, the vehicle gels to a second viscosity which is greater than the first viscosity.

For the purpose of present invention, an ophthalmically acceptable vehicle includes, but is not limited to, Sun Pharma's, formerly InSite Vision's drug delivery system, DuraSite® 2, and the terms “ophthalmically acceptable vehicle” or “vehicle” include DuraSite® 2. The vehicle may be a DuraSite® 2 composition disclosed in U.S. Pat. No. 8,501,800 to Bowman et al. Preferably, the DuraSite® 2 composition comprises: citric acid anhydrous (0.2%), sodium citrate dihydrate (0.14%), mannitol (1.1%), poloxamer 407 (0.2%), sodium hydroxide (q.s. to adjust pH 6.3), polycarbophil (0.8125%), chitosan hydrochloride (0.025%), sodium chloride (0.45%), edetate disodium dihydrate (0.1%), and water for injection (q.s. to 100%); preserved with benzalkonium chloride (0.003%)

As used herein, a “sufficient period” for treatment of blepharitis and/or ocular pain means a sufficient time to prevent, reduce, or eliminate the occurrence of clinical signs and symptoms associated with blepharitis and/or ocular pain in the eye of a subject. Such an amount of time can be assessed, for example, by evaluating eradication and/or reduction in the clinical signs or symptoms of blepharitis and/or ocular pain and the subject no longer suffers its debilitating effects. For blepharitis of a particular pathophysiological origin, the frequency, dosage, and length of time can be determined in consultation with a physician.

As used herein, “clinical signs or symptoms of blepharitis” include redness and burning sensation of the eyes, itching, gritty irritation of the eyelids, flaking of skin around the eyes, redness and swelling of the eyelids, crusted scales on the eyelashes, frothy tears, sensitivity to light, loss of eyelashes, misdirected growth of eyelashes, a greasy appearance to the eyelids, sticky secretions near the eyelashes, dry eye sensation, redness in the eyelid margins, tearing, and any combination thereof.

As used herein, the “total clinical signs and symptom score” refers to the score obtained by addition of the “individual clinical sign and symptom score” associated with blepharitis, such as eyelid swelling, eyelid redness, eyelid debris, and eyelid irritation. Wherein, the “individual clinical sign and symptom score” are evaluated and scored using a 0 to 3 grading scale. For blepharitis, the individual clinical scores (eyelid swelling, eyelid redness, eyelid debris) are evaluated and scored using a 0 to 3 grading scale (in general, 0=none, 1=mild, 2=moderate, and 3=severe). The symptom of eyelid irritation is also evaluated and graded by the participant using a 0 to 3 grading scale (0=almost none of the time—≤25% of the time; 1=occasionally—26-50% of the time; 2=frequently—51-75% of the time; 3=almost all of the time—≥76% of the time). The individual clinical sign and symptom score and the total clinical signs and symptom score of subjects are measured at each scheduled visit. For the purpose of present invention, the efficacy analysis is based on whether or not a subject's Day 15 total clinical signs and symptom score had decreased by at least 2 units from Day 1 (baseline) score, with no increase in individual sign and symptom score.

As used herein “active ingredient” refers to the primary compound responsible for reducing, preventing, or eliminating the clinical signs and symptoms of blepharitis and/or ocular pain. Exemplary active ingredients include glucocorticoids, as disclosed herein.

As used herein “an ophthalmically acceptable salt” will include those that exhibit no deleterious effects on the eye as well as being compatible with the active ingredient itself and the components of the ophthalmically acceptable vehicle. Salts or zwitterionic forms of the active ingredient glucocorticoids of the present invention can be water or oil-soluble or dispersible. The salts can be prepared during the final isolation and purification of the glucocorticoid or separately by adjusting the pH of the appropriate glucocorticoid formulation with a suitable acid or base.

In another aspect, the present invention provides a method of treating an ocular infective condition comprising administering to the affected eye of a subject an effective amount of an active ingredient in an ophthalmically acceptable vehicle for a sufficient period of time. In one embodiment, the ocular infective conditions are associated with inflammation and/or pain and such conditions may include, but are not limited to, blepharitis, post-operative ocular inflammation, post-operative ocular pain, prevention of ocular pain in subjects undergoing ocular surgery and combinations thereof.

In another embodiment, when the subject suffering from an ocular infective conditions is administered an effective amount of an active ingredient in an ophthalmically acceptable vehicle for a sufficient period of time, the subject shows a decrease in total clinical signs and symptom score at day 15 by at least 2 units from day 1 with no increase in individual clinical sign and symptom score.

In some embodiments, the disclosure provides a method of treating blepharitis that includes administering to the affected eye of a subject an effective amount of an active ingredient in an ophthalmically acceptable vehicle for a sufficient period of time to treat blepharitis.

In some embodiment, the disclosure provides a method of treating post-operative inflammation and/or post-operative ocular pain that includes administering to the affected eye of a subject an effective amount of an active ingredient in an ophthalmically acceptable vehicle for a sufficient period of time.

In yet some embodiment, the disclosure provides a method of preventing ocular pain in a subject undergoing ocular surgery, comprising administering to the affected eye of a subject an effective amount of an active ingredient in an ophthalmically acceptable vehicle for a sufficient period of time.

In another embodiment, when the subject suffering from blepharitis and/or ocular pain is administered an effective amount of an active ingredient in an ophthalmically acceptable vehicle for a sufficient period of time, the subject shows a decrease in total clinical signs and symptom score at day 15 by at least 2 units from day 1 with no increase in individual clinical sign and symptom score.

The active ingredient may consist essentially of a glucocorticoid. The ophthalmically acceptable vehicle may be one that upon contact with tear fluid it gels to a second viscosity that is greater than the first viscosity. An ophthalmically acceptable vehicle may be DuraSite® 2.

In some embodiments, an effective amount of an active ingredient is the amount used in the treatment of ocular pain and/or blepharitis and/or prophylaxis against blepharitis. This amount will achieve the goal of preventing, reducing, or eliminating blepharitis and/or ocular pain. An effective amount includes, but is not limited to, from about 0.1 μg to 100 μg per dose in one embodiment, and from about 1 μg to 10 μg per dose in another embodiment. An effective amount includes all values in between and fractions thereof, for example, about 0.1 μg, 0.5 μg, 1 μg, 5 μg, 10 μg, 15 μg, 20 μg, 30 μg, 40 μg, 50 μg, 60 μg, 70 μg, 80 μg, 90 μg, up to about 100 μg per dose. An effective amount can be administered in a dosing regimen once per day, twice per day, thrice per day, or any number of times per day and can be determined in consultation with a physician. An effective amount can be administered as a solution in eye drop form as about 0.05% to about 0.50% by weight solution of the active ingredient, including for example, about 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.25%, 0.30%, 0.35%, 0.40%, 0.45%, and about 0.50% and all values in between and fractions thereof.

In some embodiments, an active ingredient consists essentially of a glucocorticoid. Glucocorticoids are potent anti-inflammatory agents and can often be successfully administered independent of the underlying cause of inflammation. Without being bound by theory, glucocorticoids' primary anti-inflammatory mechanism are reported to be related to lipocortin-1 (annexin-1) synthesis. Lipocortin-1 suppresses phospholipase A2, thereby blocking eicosanoid production, and inhibits various leukocyte inflammatory events. In addition, glucocorticoids have been shown to suppress cyclooxygenases, including COX-1 and COX-2.

Glucocorticoids can initiate an anti-inflammatory effect by binding to the cytosolic glucocorticoid receptor (GR). After binding GR, the receptor-ligand complex translocates to the cell nucleus, where it can bind to glucocorticoid response elements (ORE) in the promoter region of target genes. The proteins encoded by these upregulated genes have a wide range of effects including anti-inflammatory effects mediated, for example, by lipocortin-1 as described above. Glucocorticoids can also reduce the transcription of pro-inflammatory genes by a mechanism of transrepression. Thus, inflammation associated with blepharitis can be ameliorated by glucocorticoid treatment.

Accordingly, in some embodiments, the active ingredient of the compositions and methods of the invention consists essentially of a glucocorticoid including, for example, hydrocortisone, cortisone acetate, prednisone, prednisolone, methylprednisolone, dexamethasone, betamethasone, triamcinolone, beclomethasone, and fluorometholone. Other glucocorticoids useful in the method for treating blepharitis include, for example, 21-acetoxypregnenolone, alclometasone, algestone, amcinonide, budesonide, chloroprednisone, clobetasol, clobetasone, clocortolone, cloprednol, corticosterone, cortisone, cortivazol, deflazacort, desonide, desoximetasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate, hydrocortarnate, loteprednol etabonate, mazipredone, medrysone, meprednisone, mometasone furoate, paramethasone, prednicarbate, prednisolone 25-diethylamino-acetate, prednisolone sodium phosphate, prednival, prednylidene, rimexolone, tixocortol, triamcinolone acetonide, triamcinolone benetonide, triamcinolone hexacetonide, their ophthalmically acceptable salts, combinations thereof, and mixtures thereof. In one embodiment, the glucocorticoid includes dexamethasone, prednisone, prednisolone, methylprednisolone, medrysone, triamcinolone, loteprednol etabonate, ophthalmically acceptable salts thereof, combinations thereof, and mixtures thereof.

The effects of treating blepharitis and/or ocular pain with dexamethasone, in particular, with the aid of the slow-release ophthalmically acceptable vehicle, are shown in the Example below, although any of the aforementioned glucocorticoids are believed to be useful in the treatment of blepharitis and/or ocular pain in accordance with the instant invention. In accordance with various embodiments of the invention, dexamethasone includes, for example, dexamethasone sodium phosphate, dexamethasone (alcohol), dexamethasone acetate, dexamethasone dimethylbutyrate, dexamethasone trimethylacetate, dexamethasone dipropionate, dexamethasone acefurate, and mixtures thereof.

In some embodiments, the glucocorticoid is present in a range from about 0.05% and to about 0.5% by weight, while in other embodiments the glucocorticoid is present in a range from about 0.08% to about 0.12% by weight. The amount of glucocorticoid based on weight percent can be any value between these values, including, for example, 0.05%, 0.060%, 0.07%, 0.08%, 0.09%, 0.10%, 0.11%, 0.12%, 0.13%, 0.14%, 0.15%, 0.16%, 0.17%, 0.18%, 0.19%, 0.20%, 0.25%, 0.30%, 0.35%, 0.40%, 0.45%, and about 0.50% by weight and all values in between and fractions thereof. A standard solution of dexamethasone, in particular, for ophthalmic use is about 0.10% by weight.

In one aspect, the present invention provides a method of treating blepharitis, comprising administering to the affected eye of a subject a composition comprising 0.1% w/w of dexamethasone in an ophthalmically acceptable vehicle, wherein the subject shows a decrease in total clinical signs and symptom score at day 15 by at least 2 units from day 1 with no increase in individual clinical sign and symptom score. In another embodiment, the composition is administered for at least one to four times a day for a period of at least one to six weeks or longer. In a preferred embodiment, the administration is two times a day for at least 14 days. In one embodiment, the blepharitis is active, symptomatic blepharitis.

In another aspect, the present invention provides a composition comprising 0.1% w/w of dexamethasone in an ophthalmically acceptable vehicle for use in the treatment of blepharitis. In one embodiment, the blepharitis is active, symptomatic blepharitis. In yet another embodiment, the composition comprising 0.1% w/w of dexamethasone is administered to a subject suffering from blepharitis two times a day for at least 14 days, wherein the subject shows a decrease in total clinical signs and symptom score at day 15 by at least 2 units from day 1 with no increase in individual clinical sign and symptom score.

In another aspect, the present invention provides a method of treating post-operative inflammation, comprising administering to the affected eye of a subject a composition comprising 0.1% w/w of dexamethasone in an ophthalmically acceptable vehicle, wherein the subject shows a decrease in total clinical signs and symptom score at day 15 by at least 2 units from day 1 with no increase in individual clinical sign and symptom score. In another embodiment, the composition is administered for at least one to four times a day for a period of at least one to six weeks or longer. In a preferred embodiment, the administration is two times a day, one day prior surgery, on the day of surgery and 14 days post-surgery.

In yet another aspect, the present invention provides a method of preventing ocular pain in subject undergoing ocular surgery comprising administering to the affected eye of a subject a composition comprising 0.1% w/w of dexamethasone in an ophthalmically acceptable vehicle, wherein the composition is administered for at least one to four times a day for a period of at least one to six weeks or longer. In a preferred embodiment, the administration is two times a day, one day prior surgery, on the day of surgery and 14 days post-surgery.

In another aspect, the present invention provides a composition comprising 0.1% w/w of dexamethasone in an ophthalmically acceptable vehicle for use in the prevention of ocular pain in the subject undergoing ocular surgery. In one embodiment, the blepharitis is active, symptomatic blepharitis. In yet another embodiment, a composition comprising 0.1% w/w of dexamethasone is administered to a subject undergoing ocular surgery for at least two times a day, one day prior surgery, on the day of surgery and 14 days post-surgery. In one embodiment, the subject is undergoing ocular surgery, when administered said composition, and the subject shows a decrease in total clinical signs and symptom score at day 15 by at least 2 units from day 1 with no increase in individual clinical sign and symptom score.

In some embodiments, the disclosure is directed to a kit which comprises: (a) a composition comprising about 0.1% by weight dexamethasone in an ophthalmically acceptable vehicle capable of slow release as detailed herein and, optionally, (b) instructions for using the composition of (a) for the treatment of blepharitis and/or ocular pain.

In some embodiments, the kit further includes a delivery device for administering the composition. In some embodiments, the delivery device for administering can include a bottle, dropper, cup, specialized eye-wash apparatus, wetted towel or sponge. In some embodiments, the kit includes a cleaning apparatus (e.g., a towel, pad, cloth, brush, sponge, etc.) and/or a cleaning solution (e.g., purified water, a detergent solution, a boric acid solution, etc.). In some embodiments of the present disclosure, the ocular area is cleaned prior to administration of the composition of the present invention.

The composition can be individually packaged for a single dose administration; e.g., in a bottle, jar, ampoule, tube, syringe, envelope, container, or vial. When the composition is individually packaged, in some embodiments, the composition does not include a preservative. Alternatively, the composition can be contained in a package that is capable of holding multiple units; e.g., in resealable glass or plastic packages. In some kits, the components of the composition are mixed together immediately preceding their usage. For example, in some embodiments, one or more dry components of the composition of the kit are packaged in a separate container; e.g., a plastic bottle, and then mixed with one or more of the liquid components of the composition immediately prior to use. Optionally, the kit of the present invention can include a dropper or other device for transferring or administering the composition to a subject.

The kit can further include instructions for using the composition of the present invention. For example, such instructions can be in a form prescribed by a governmental agency regulating the manufacture, use or sale of pharmaceuticals or biological products, which reflects approval by the agency of the manufacture, use or sale for human application. In some embodiments, the kit further includes information on the use of the composition or a pre-recorded media device which, e.g., provides information on the use of the method of the present invention.

The kit can also include a container for storing the components of the kit. The container can be, for example, a bag, box, envelope or any other container suitable for use in the present invention. In some embodiments, the container is large enough to accommodate each component of the present invention. However, in some cases, it can be desirable to have a smaller container which is large enough to carry only some of the components.

In one embodiment, the ophthalmically acceptable vehicle according to any of the aspects/embodiments of the present disclosure may be the one defined earlier and that the said ophthalmically acceptable vehicle, upon contact with tear fluid gels to a second viscosity that is greater than the first viscosity. In a preferred embodiment, the ophthalmically acceptable vehicle is DuraSite® 2.

It is understood that modifications which do not substantially affect the activity of the various embodiments of this invention are also included within the disclosure provided herein. Accordingly, the following examples are intended to illustrate but not limit the present disclosure.

### EXAMPLES

### Reference Example 1: Test and Control Compositions

This Example shows composition which are used in the treatment of blepharitis and/or ocular pain.

| | Concentration | Amount Per |
|---|---|---|
| Ingredient | (% w/w) | g (mg) |
| Dexamethasone, USP, | 0.100 | 1.00 |
| EP, Micronized | | |
| Mannitol, USP | 1.100 | 11.00 |
| Citric Acid Anhydrous, USP | 0.200 | 2.00 |
| Sodium Citrate Dihydrate, USP | 0.140 | 1.40 |
| Poloxamer 407, NF | 0.200 | 2.00 |
| Benzalkonium Chloride, NF | 0.003 | 0.03 |
| Polycarbophil, USP | 0.8125 | 8.125 |
| Chitosan Hydrochloride | 0.025 | 0.25 |
| Sodium Chloride, USP | 0.450 | 4.50 |
| Edetate Disodium Dihydrate, USP | 0.100 | 1.00 |
| Sodium Hydroxide, 2N, NF | Adjust to pH 6.3 | Adjust to pH 6.3 |
| Water for Injection, USP | qs to 100% | qs to 1 g |

The control composition is a vehicle composition, as disclosed earlier in the current specification or in U.S. Pat. No. 8,501,800 assigned to Bowman et al., the entire contents of which are incorporated herein by reference.

The following examples relate to the efficacy and safety of compositions containing dexamethasone in DuraSite® 2 as compared to administration of DuraSite® 2 alone in the treatment of subjects with active, symptomatic blepharitis.

### Example 1

This example shows a composition of 0.1% dexamethasone in DuraSite® 2 is useful for treating blepharitis.

In this example, administration of a composition containing 0.1% dexamethasone in DuraSite® 2 or administration of a control containing only DuraSite® 2 was performed. Administration was performed twice daily for two weeks.

For the dexamethasone-containing compositions, the composition was administered as a topical ophthalmic formulation of 0.1% dexamethasone in DuraSite® 2 vehicle (Sun Pharma's, formerly InSite Vision's drug delivery system) twice daily (one drop in the morning and one drop in the evening) for 14 days. For the control compositions, the vehicle (DuraSite® 2) was administered as a matching topical ophthalmic formulation without dexamethasone twice daily (one drop in the morning and one drop in the evening) for 14 days.

In this Example, 558 subjects were enrolled and 537 subjects completed the study. Of those, 368 subjects were enrolled and administered the dexamethasone-containing formulation (352 completed) and 190 subjects were enrolled and administered the control (185 completed). Of those subjects that did not complete the study, 4 did so because of an adverse event (4 in the dexamethasone group/0 in the control group), 1 did so because of a physician's decision (I/O), 6 did so because of a lack of efficacy (3/3), 5 were lost to follow-up (5/0), and 3 were lost for various administrative reasons (1/2).

### Primary Outcome Measurement—Reduction of Symptoms

For the primary outcome measurement, the clinical signs of blepharitis (eyelid swelling, eyelid redness, eyelid debris) were evaluated and scored using a 0 to 3 grading scale (in general, 0=none, 1=mild, 2=moderate, and 3=severe). The symptom of eyelid irritation was also evaluated and graded by the participant using a 0 to 3 grading scale (0=almost none of the time—≤25% of the time; 1=occasionally—26-50% of the time; 2=frequently—51-75% of the time; 3=almost all of the time—≥76% of the time). At each measurement time, the total clinical signs and symptom score was obtained by adding each of the individual scores for each domain (eyelid swelling, eyelid redness, eyelid debris, and eyelid irritation).

Participants with a decrease in total clinical signs and symptom score at Day 15 by at least 2 units from Day 1 (baseline) with no increase in any sign and symptom were defined as Responders, and those not meeting these criteria were defined as Non-responders.

TABLE 1

| | 0.1% dexamethasone in | | | |
|---|---|---|---|---|
| | DuraSite ® 2 | | DuraSite ® 2 | |
| Participants Analyzed | 353 | | 185 | |
| Responders | 276 | (78.19%) | 129 | (69.73%) |
| Non-Responders | 77 | (21.81%) | 56 | (30.27%) |

As shown in Table 1, the subject population that was administered the composition including dexamethasone in DuraSite® 2 had a greater percentage of its participants exhibit improvement in their clinical signs and symptoms.

### Secondary Outcome Measurement—Complete Resolution of Eyelid Irritation

For the secondary outcome measurement, the complete resolution of eyelid irritation, which was defined as eyelid irritation with grading of 0, at Day 15 was analyzed. The symptoms of eyelid irritation were graded by the participant using a 0 to 3 grading scale, where 0=almost none of the time—≤25% of the time; 1=occasionally—26-50% of the time; 2=frequently—51-75% of the time; 3=almost all of the time—≥76% of the time.

TABLE 2

| | 0.1% dexamethasone in | | |
|---|---|---|---|
| | DuraSite ® 2 | DuraSite ® 2 | |
| Participants Analyzed | 368 | 190 | |
| With Complete Resolution | 127 (34.51%) | 52 | (27.37%) |
| Without Complete | 241 (65.49%) | 138 | (72.63%) |
| Resolution | | | |

As shown in Table 2, the subject population that was administered the composition including dexamethasone in DuraSite® 2 had a greater percentage of its participants exhibit complete resolution of eyelid irritation.

We claim:

1. A method of treating blepharitis, comprising administering to an affected eye of a subject a composition comprising 0.1% w/w of dexamethasone in an ophthalmically acceptable vehicle, wherein the subject shows a decrease in total clinical signs and symptom score at day 15 by at least 2 units from day 1 with no increase in individual clinical sign and symptom score.

2. The method according to claim 1, wherein the blepharitis is active, symptomatic blepharitis.

3. The method according to claim 1, wherein the composition is administered two times a day for at least 14 days.

4. A composition comprising 0.1% w/w of dexamethasone in an ophthalmically acceptable vehicle for use in the treatment of blepharitis.

5. The composition according to claim 4, wherein the composition is administered to a subject suffering from blepharitis two times a day for at least 14 days.

6. The composition according to claim 5, wherein the subject shows a decrease in total clinical signs and symptom score at day 15 by at least 2 units from day 1 with no increase in individual clinical sign and symptom score.

7. The composition according to claim 4, wherein the blepharitis is active, symptomatic blepharitis.

8. A kit comprising: (a) a composition comprising about 0.1% w/w of dexamethasone in an ophthalmically acceptable vehicle; and (b) instruction for using the said composition for the treatment of blepharitis, ocular pain or both.

9. The kit according to claim 8, wherein the kit further comprises a delivery device for administering the composition, a cleaning apparatus and a cleaning solution.

10. The kit according to claim 9, wherein the delivery device for administering the composition is selected from a bottle, a dropper, a cup, specialized eye-wash apparatus, wetted towel and sponge.

11. The kit according to claim 9, wherein the cleaning apparatus is selected from a towel, pad, cloth, brush and sponge.

12. The kit according to claim 9, wherein the cleaning solution is selected from purified water, a detergent solution and a boric acid solution.

13. A method of preventing ocular pain in a subject undergoing ocular surgery, comprising administering to an affected eye of a subject a composition comprising 0.1% w/w of dexamethasone in an ophthalmically acceptable vehicle.

14. The method according to claim 13, wherein the subject shows a decrease in total clinical signs and symptom score at day 15 by at least 2 units from day 1 with no increase in individual clinical sign and symptom score.

15. The method according to claim 13, wherein the composition is administered two times a day, one day prior surgery, on the day of surgery and 14 days post-surgery.

16. A method of treating an ocular infective condition comprising administering to an affected eye of a subject an effective amount of an active ingredient in an ophthalmically acceptable vehicle for a sufficient period of time, wherein the subject shows a decrease in total clinical signs and symptom score at day 15 by at least 2 units from day 1 with no increase in individual clinical sign and symptom score.

17. The method according to claim 16, wherein the ocular infective condition is associated with inflammation, pain or both and is selected from blepharitis, post-operative ocular inflammation, post-operative ocular pain, ocular pain in patients undergoing ocular surgery and combinations thereof.

18. The method according to claim 16, wherein the active ingredient consists essentially of a glucocorticoid.

19. The method according to claim 16, wherein the effective amount of the active ingredient is 0.1% w/w of the active ingredient.

20. A method of treating blepharitis, comprising administering to an affected eye of a subject an effective amount of an active ingredient in an ophthalmically acceptable vehicle for a sufficient period of time, wherein the subject shows a decrease in total clinical signs and symptom score at day 15 by at least 2 units from day 1 with no increase in individual clinical sign and symptom score.

21. The method according to claim 20, wherein the blepharitis is active, symptomatic blepharitis.

22. The method according to claim 20, wherein the active ingredient in the ophthalmically acceptable vehicle is administered two times a day for at least 14 days.

23. The method according to claim 20, wherein the active ingredient consists essentially of a glucocorticoid.

24. The method according to claim 20, wherein the effective amount of the active ingredient is 0.1% w/w of the active ingredient.

25. A method of preventing ocular pain in a subject undergoing ocular surgery, comprising administering to an affected eye of a subject an effective amount of an active ingredient in an ophthalmically acceptable vehicle for a sufficient period of time, wherein the subject shows a decrease in total clinical signs and symptom score at day 15 by at least 2 units from day 1 with no increase in individual clinical sign and symptom score.

26. The method according to claim 25, wherein the active ingredient in the ophthalmically acceptable vehicle is administered two times a day, one day prior surgery, on the day of surgery and 14 days post-surgery.

27. The method according to claim 25, wherein the active ingredient consists essentially of a glucocorticoid.

28. The method according to claim 25, wherein the effective amount of the active ingredient is 0.1% w/w of the active ingredient.
